(19) United States
(12) Patent Application Publication
(10) Pub. No.: US 2007/0016015 A1
(43) Pub. Date: Jan. 18, 2007

(54) Method for the visualization of a vscular insert

(75) Inventors: Estelle Camus, Erlangen (DE); Markus Lendl, Ottensoos (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/486,696

(22) Filed: Jul. 14, 2006

(30) Foreign Application Priority Data
Jul. 15, 2005 (DE) 10 2005 033 235.8

(51) Int. Cl. A61B 5/05

(52) U.S. Cl. 600/427

(57) ABSTRACT

Method for the visualization of a vascular insert comprises: performing an x-ray with a contrast agent and digital subtraction angiography prior to implantation of the vascular insert, simultaneous performing an electrocardiogram or recording a blood pressure signal while performing the x-ray, performing a further x-ray without contrast agent after implantation of the vascular insert, simultaneous performing an electrocardiogram or recording a blood pressure signal while performing the further x-ray, selecting pairs of x-ray images, comprising in each case one taken prior to implantation and one after the implantation, overlaying the two x-ray images in order to visualize the vascular insert.

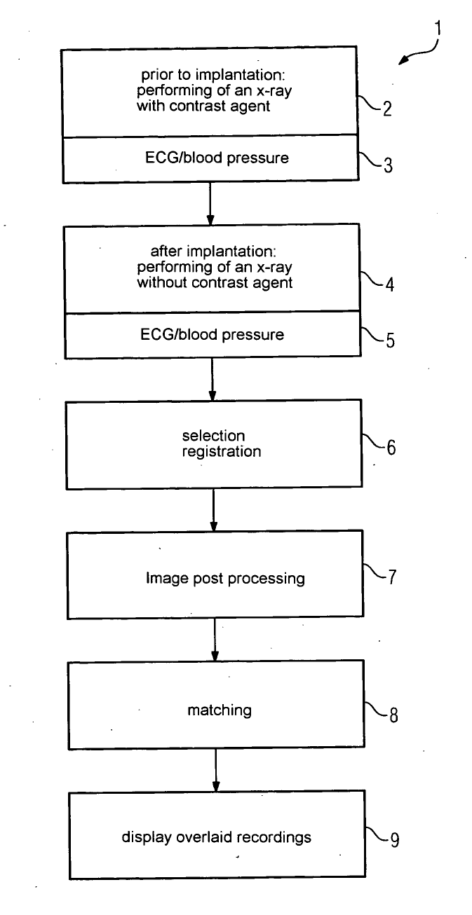

### CROSS REFERENCE TO RELATED APPLICATIONS

This application claims priority of German application No. 10 2005 033 235.8 filed Jul. 15, 2005, which is incorporated by reference herein in its entirety.

### FIELD OF THE INVENTION

The invention relates to a method for visualizing a vascular insert in a patient in an x-ray image. The invention further relates to a method for visualizing the vascular insert and the surrounding vessel anatomy of the vascular insert in the x-ray image at the same time.

### BACKGROUND OF THE INVENTION

Vascular inserts, for example stents or artificial valves, are introduced into the body, for example into a coronary vessel, in the course of vascular interventions. A stent is an insertable prosthesis resembling an expanding trellis, which expands autonomously after implantation. Stents are also used to bridge or maintain the lumen in the case of stenosis or obstructions of hollow organs or in the event of vascular stenosis caused by arterial sclerosis.

In practice, the problem arises that after implantation of a stent, it is not possible to represent its structure and thus its degree of expansion or its position on x-ray projections with sufficient precision. This is on the one hand attributable to the very delicate structures of the stent as well as to the fact that the vessels, for example the coronary vessels, as well as the stent itself, are in constant motion due to the beating of the subject's heart and their breathing. The temporal and spatial resolution of traditional X-ray machines is insufficient to represent the implanted stent with sufficient visual clarity without artifacts.

Methods have already been proposed by means of which the visualization of stents may be improved. In U.S. Pat. No. 5,457,728 a method is proposed, by means of which a number of X-ray projections of stents can be recorded. A relevant region of interest (ROI) around the stent is then selected, and the image data in this area summed. This is intended to improve the signal-to-noise ratio, so that the perceptibility of the stent and its fine structures is thereby enhanced. In addition, the region of interest is represented without movement, in that it appears in the same position in each image, while the remainder of the image moves. Although as the method is based on x-ray images, the stent can be visualized, this does not apply to the anatomy of the vessels surrounding the stent.

### SUMMARY OF THE INVENTION

The present invention thus takes as its basis the problem of defining a method for the visualization of a vascular insert, in which the vascular insert and the associated anatomy can be represented at the same time.

To solve this problem a method of the type mentioned in the introduction involving the following steps is provided:
a) Prior to implantation of the vascular insert:
Performing of an x-ray with a contrast agent and digital subtraction angiography,
b) Simultaneous performing of an electrocardiogram and/or recording of a blood pressure signal,
c) After implantation of the vascular insert:
Performing of an x-ray without contrast agent,
d) Simultaneous performing of the electrocardiogram and/or recording of a blood pressure signal,
e) Selection of pairs of x-ray images, comprising in each case one taken prior to implantation in step a) and one after implantation in step c),
f) Overlaying of the two x-ray images in order to visualize the vascular insert.

In the method according to the invention, the recording of the angiographic images, that is the x-rays, consist of two phases. In step a) angiographic images are recorded after administering a contrast agent with digital subtraction angiography, in order to represent the anatomy of the diseased vessel to be examined. This is followed by implantation of the stent. After implantation of the stent, a further x-ray may be taken with contrast agent.

Angiographic images are then obtained without contrast agent, in order to visualize the implanted vascular insert. The method according to the present invention further provides for the performing of an electrocardiogram and/or a blood pressure signal, so that x-rays taken in steps a) and c) are selected which have been obtained in the same phase of the cardiac rhythm. This serves to avoid artifacts caused by the subject's heartbeat and breathing. On the basis of the ECG and /or blood pressure signal recording, two x-ray images taken in steps a) and c) are selected and superimposed one over the other. The overlaid image accordingly shows both the vascular insert and the anatomy of the vessel. In this way, the method according to the invention enables improved visualization of the position and state of expansion of a vascular insert, relative to the anatomy of the vessel. With the method according to the invention, the success of the implantation of the vascular insert as performed can be assessed significantly more effectively than with customary visualization techniques.

The method according to the present invention is particularly suitable when the vascular insert takes the form of a stent or an artificial valve such as a heart valve, since correct positioning is of particular importance in the case of such vascular inserts.

In the method according to the present invention it can be provided for that the two x-ray images selected in step e) are registered with each other on the basis of the electrocardiograms or based on the blood pressure signals. To do so, each x-ray is simultaneously assigned to the corresponding ECG signal or blood pressure signal. In selecting the x-ray images it should be ensured that in each case only those x-rays are selected which have been obtained during the same phase of the cardiac rhythm.

In the method according to the present invention, at least one, and preferably both of the x-rays selected in step e) can be subjected to post-processing of the image. The post-processing is intended to improve the perceptibility of the vascular insert, and especially of the stent. The post-processing can in particular encompass improvement of the signal-to-noise ratio and/or an image summation and/or an edge enhancement.

Insofar as the method according to the present invention includes the performing of an image summation, a matching process is required to overlay the two x-ray images. By matching the two images, it is possible to correct any possible displacements or rotations between the two images. It is suitable to perform the matching by means of rotation and/or displacement and/or stretching and/or diminution, in order to compensate for any divergences between the two images caused by the patient's breathing or their movement. Here, the matching can either be performed automatically by means of a software program, on the basis of anatomical and/or artificial landmarks, or carried out manually.

If the two x-rays are represented one overlaid over the other, the x-ray image obtained in step a) using a contrast agent can be displayed in inverted form, thus improving visualization of the vascular insert.

### BRIEF DESCRIPTION OF THE DRAWINGS

Further advantages of details of the present invention are explained on the basis of an embodiment with reference to the figure which shows a flow chart of the individual steps in the method according to the invention.

### DETAILED DESCRIPTION OF THE INVENTION

The figure shows a flow chart of the individual steps in the method according to the invention.

In the case of method 1 for the visualization of a vascular insert, prior to implantation an x-ray 2 is taken, with contrast agent and digital subtraction angiography. In this exemplary embodiment, the x-ray image shows a diseased coronary artery. An ECG signal 3 or a blood pressure signal is obtained synchronously with this. Both signals can be used to assign the x-ray image 2 to the particular phase of the heartbeat. After implantation of the stent, an x-ray 4 can be taken without contrast agent. The configuration of the x-ray system should here be unaltered by comparison with x-ray 2, that is to say the position of the patient relative to the x-ray system and the angulation should be unchanged. X-ray image 4 is selected such that visibility of the stent is as complete as possible. At the same time, an ECG signal 5 or blood pressure signal is also obtained with x-ray 4.

The next step involves selection 6 of an x-ray image 2, which displays the anatomy of the vessel in a clear manner. Selection 6 may be performed manually, though it is also possible to use a software program to select a particularly suitable x-ray image based on prescribed criteria. An x-ray image 4 obtained after the implantation and taken during the same phase of the cardiac rhythm as the selected x-ray image 2 is subsequently selected. This automatically initiates temporal registration.

Selection 6 and the registration are then followed by post-processing of the image 7, in which an image summation is performed. An edge enhancement is likewise carried out, in order to improve the signal-to-noise ratio in the region of the stent and to enhance visibility of the stent.

By matching 8 the images, any possible displacement, stretching or rotation of the x-rays is corrected.

In step 9, the two x-ray images, one obtained with a contrast agent and one without, are overlaid one over the other. In this case the image obtained from the x-ray 4 with contrast agent is represented in inverted form.

1-8. (canceled)

9. A method for visualizing a vascular insert in a patient, comprising:
recording an x-ray image of a vessel anatomy to be implanted with the vascular insert with a contrast agent and digital subtraction angiography using an x-ray device prior to the implantation of the vascular insert into the patient;
simultaneously taking an electrocardiogram or recording a blood pressure signal of the patient while recording the x-ray image;
implanting the vascular insert;
recording a further x-ray image of the vessel anatomy containing the vascular insert without contrast agent after the implantation of the vascular insert using the x-ray device;
simultaneous taking a further electrocardiogram or recording a further blood pressure signal of the patient while recording the further x-ray image;
selecting a pair of x-ray images from the x-ray images taken prior to the implantation and after the implantation; and
overlaying the pair of x-ray images in order to visualize the vascular insert.

10. The method as claimed in claim 9, wherein the vascular insert is a stent or an artificial valve.

11. The method as claimed in claim 9, wherein the selected x-ray images are registered with each other based on the electrocardiogram or the blood pressure signals.

12. The method as claimed in claim 9, wherein one or both of the selected x-ray images is subjected to an image post-processing to improve a perceptibility of the vascular insert.

13. The method as claimed in claim 12, wherein the image post-processing encompasses an improvement of a signal to noise ratio, an image summation, or an edge enhancement.

14. The method as claimed in claim 9, wherein a matching of the selected x-ray images is performed by stretching, rotation, or displacement prior to the overlaying step.

15. The method as claimed in claim 14, wherein the matching is performed based on an anatomical or an artificial landmark of the vessel anatomy.

16. The method as claimed in claim 9, wherein the x-ray image taken prior to the implantation is represented in an inverted form in the overlaying.

17. The method as claimed in claim 9, wherein the x-ray images taken prior to the implantation and after the implantation are taken at an identical patient position relative to the x-ray device and an identical setting of the x-ray device.

18. A device for visualizing a vascular insert in a patient, comprising:
an x-ray device that:
takes an x-ray image of a vessel anatomy to be implanted with the vascular insert with a contrast agent and digital subtraction angiography prior to the implantation of the vascular insert into the patient,
takes a further x-ray image of the vessel anatomy containing the vascular insert without contrast agent after the implantation of the vascular insert;
an electrocardiogram device for performing an electrocardiogram when taking the x-ray images;
a blood pressure measurement device for recording a blood pressure signal when taking the x-ray images; and
a calculating device for selecting a pair of x-ray images from the x-ray images taken prior to the implantation and after the implantation and for overlaying the pair of x-ray images.
